(19) United States
(12) Patent Application Publication
(10) Pub. No.: US 2006/0223786 A1
(43) Pub. Date: Oct. 5, 2006

(54) Transdermal pain control method and device

(76) Inventors: David J. Smith, San Diego, CA (US); Mark j. Badria, San Diego, CA (US); Ollen Williams JR., El Cajon, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/097,878

(22) Filed: Apr. 1, 2005

(51) Int. Cl. A61K 31/66; A61K 31/573; A61K 31/542; A61K 31/485; A61K 31/445; A61K 31/192; A61K 31/165; A61K 31/137; A61K 31/13

(52) U.S. Cl. 514/114; 514/171; 514/282; 514/317; 514/617; 514/663; 514/649; 514/569; 514/570

(57) ABSTRACT

Compositions comprising skin-permeable pharmaceutically effective amounts of an opioid agonist; an N-methyl-D-aspartate receptor antagonist and an anti-inflammatory may be incorporated into formulations and devices suitable for transdermal delivery of the active ingredients to alleviate pain.

### BACKGROUND OF THE INVENTION

1. Field of the Invention

This invention relates to compositions useful for alleviating pain and methods for their delivery to humans. More particularly, this invention relates to compositions comprising an opioid agonist, an NMDA receptor antagonist, and an anti-inflammatory, and methods for the transdermal delivery of those compositions to relieve pain.

2. Description of the Related Art

The treatment of physical pain concerns health care professionals throughout the world. The treatment of chronic pain is particularly challenging because of the frequent need for repeated administration of pain relief medication. Chronic pain is generally considered to be pain that continues a month or more beyond the usual recovery period for an illness or injury or pain that goes on over months or years as a result of a chronic condition. It may be continuous or come and go. It is estimated that chronic pain disables, to some degree, about 86 million Americans. It is regarded as a source of frustration for the health care professionals who care for the patient, and affects the quality of life and economic security not only of the person with pain, but also his or her family. It is estimated that United States business and industry loses about $90 billion annually to sick time, reduced productivity, and direct medical and other benefit costs due to chronic pain among employees. In some cases, repeated administration of the pain relief medication causes sufferers of chronic pain to develop an undesirable tolerance or addiction, creating further health issues for the patient and additional challenges for the health care professional.

There are a number of methods for administering pain relief medications, including oral and parenteral (administered in a manner other than through the digestive tract). Oral administration is most frequently accomplished by formulating the pain relief medication into tablet or syrup and allowing the patient to swallow it. This method is simple, well accepted and relatively painless, but may be problematic for uncooperative patients. Also, there is often a considerable lapse of time between administration of the pain relief medication and its therapeutic effect because of the time needed for gastrointestinal absorption. This time lag is of particular concern when a patient is suffering from severe or chronic pain. Faster administration may be accomplished by direct injection of the pain relief medication, but most people consider the injection itself to be painful and thus undesirable. What is needed is a method for administering a variety of pain relief formulations that is fast, well tolerated and relatively painless.

### SUMMARY OF THE INVENTION

The invention is directed towards pain relief delivery systems and methods and, in particular, pain relief compositions that comprise a pain relief drug and methods of delivering such compositions transdermally for the relief of pain.

Preferred pain relief drugs are opioid agonists. To inhibit the development of tolerance and/or addiction to the opioid agonists by the patient, preferred drug combinations further comprise a substance that blocks the N-methyl-D-aspartate receptor, herein referred to as an “NMDA receptor antagonist.” Preferred drug combinations also comprise an anti-inflammatory drug, preferably a non-steroidal anti-inflammatory drug (NSAID). Contrary to the disclosure of U.S. Patent Application Publication 2003/0199439 A1, preferred drug combinations need not contain a α3β4 nicotinic receptor antagonist. Thus, preferred pain relief compositions are preferably free of a pharmaceutically effective amount of an α3β4 nicotinic receptor antagonist.

A preferred embodiment provides a pain relief composition comprising pharmaceutically effective amounts of (a) an opioid agonist; (b) an N-methyl-D-aspartate receptor antagonist different from the opioid agonist; and (c) an anti-inflammatory, the anti-inflammatory being different from the opioid agonist and different from the N-methyl-D-aspartate receptor antagonist; wherein the opioid agonist, the N-methyl-D-aspartate receptor antagonist and the anti-inflammatory are each in a skin-permeable form; and wherein the composition is free of a pharmaceutically effective amount of an α3β4 nicotinic receptor antagonist. Preferably, the opioid agonist is selected from the group consisting of fentanyl and sufentanil, the N-methyl-D-aspartate receptor antagonist is dextromethorphan, and the anti-inflammatory is ketorolac.

Another preferred embodiment provides a transdermal delivery device comprising the composition described above and means for delivering the composition transdermally to a human.

Another preferred embodiment provides a transdermal delivery patch comprising pharmaceutically effective amounts of (a) an opioid agonist; (b) an N-methyl-D-aspartate receptor antagonist different from the opioid agonist; and (c) an anti-inflammatory, the anti-inflammatory being different from the opioid agonist and different from the N-methyl-D-aspartate receptor antagonist; wherein the opioid agonist, the N-methyl-D-aspartate receptor antagonist and the anti-inflammatory are each in a skin-permeable form; and wherein the patch is free of a pharmaceutically effective amount of an α3β4 nicotinic receptor antagonist. Preferably, the transdermal delivery patch comprises a reservoir that contains an opioid agonist selected from the group consisting of fentanyl and sufentanil, dextromethorphan as an N-methyl-D-aspartate receptor antagonist, and ketorolac as an anti-inflammatory.

Another preferred embodiment provides a method for treating pain, comprising identifying a human suffering from pain and applying a transdermal delivery patch as described above to the human.

These and other embodiments are described in greater detail below.

### BRIEF DESCRIPTION OF THE DRAWINGS

These and other aspects of the invention will be readily apparent from the following description and from the appended drawings (not to scale), which are meant to illustrate and not to limit the invention, and in which:

FIG. 1 is a plot of illustrating the flux of fentanyl through human cadaver epidermis as a function of time.

FIG. 2 is a plot of illustrating the flux of fentanyl through human cadaver epidermis and an EVA membrane as a function of time.

FIG. 3 is a plot of illustrating the flux of fentanyl, ketorolac tromethamine and dextromethorphan from an embodiment of a reservoir patch through human cadaver epidermis as a function of time.

### DETAILED DESCRIPTION OF THE PREFERRED EMBODIMENTS

Preferred pain relief compositions comprise pharmaceutically effective amounts of (a) an opioid agonist; (b) an N-methyl-D-aspartate receptor antagonist different from the opioid agonist; and (c) an anti-inflammatory, the anti-inflammatory being different from the opioid agonist and different from the N-methyl-D-aspartate receptor antagonist. To facilitate preferred transdermal administration, the opioid agonist, the N-methyl-D-aspartate receptor antagonist and the anti-inflammatory are preferably each in a skin-permeable form. In addition, the preferred compositions are free of a pharmaceutically effective amount of an α3β4 nicotinic receptor antagonist. The term “pain” is used herein to refer to the condition to which the patient is subject and thus includes associated inflammation. It will be understood that a patient's pain is commonly, and in fact usually, associated with and resulting from inflammation at the site of the dysfunction, trauma, chronic disease or the like.

The term “opioid agonist” is used herein in the ordinary sense and thus includes opiates, opiate derivatives, opioids, and other substances whose effects are mediated by the same receptor, including mixtures thereof. Non-limiting examples of suitable opioid agonists include: alfenanil; allylprodine; alphaprodine; anileridine; benzitramide; benzylmorphine; beta-endorphin; buprenorphine; butorphanol; carfentanil; clonitazene; codeine; cyclazocine; cyclozine, desomorphine; dextromoramide; dezocine; diamorphine; diampromide; dihydromorphine; dimenoxadol; fentanyl; sufentanil; lofentanil; morphine; normorphine; dihydrocodeine; levorphanol; oxycodone; oxycodone; propoxyphene; meperidine; methadone; normethadone; meptazinol; nicomorphine; pentazocine, remifentanil, heroin, morphine-6-glucuronide; nalbuphine; meptazinol; pethidine; hydromorphone; piritramide; nicomorphine; tilidine; tramadol; opium; met-enkaphalin; delta-enkephalin; dynorphin A; peptide F; Leu-enkephalin; N-alpha-acetylmethadone; dihydromorphine; etorphine; oxymorphone; and pharmaceutically acceptable acids, bases and salts thereof. Non-limiting examples of particularly preferred opioid agonists include fentanyl, hydromorphone, hydrocodone, ketamine, methadone, oxycodone, oxymorphone, propoxyphene, sulfentanil, and pharmaceutically acceptable salts thereof.

NMDA receptor antagonists are substances known to those skilled in the art that block the N-methyl-D-aspartate receptor or that block a major intracellular consequence of NMDA receptor activation, see U.S. Pat. Nos. 5,321,012; 5,654,281 and 5,869,498, all of which are hereby incorporated by reference in their entireties, and particularly for the purpose of describing NMDA receptor antagonists and their uses. The NMDA receptor antagonist may be a mixture. Non-limiting examples of preferred NMDA receptor antagonists include amantadine, amitriptyline, D,L-2-amino-5-phosphono valeric acid, dextromethorphan, ketamine, methadone, and pharmaceutically acceptable salts thereof. Since some substances, e.g., ketamine and methadone, may be classified as both opioid agonists and NMDA receptor antagonists, it is understood that the opioid agonist in any formulation is different from the NMDA receptor antagonist.

The term “anti-inflammatory” refers to a broad class of agents useful for reducing and/or preventing inflammation, and thus includes steroidal anti-inflammatories and non-steroidal anti-inflammatories (NSAIDS). Examples of steroidal anti-inflammatories include corticosteroids such as alcometasone, clocortolone, dexamethasone, hydrocortisone, hydrocortisone 21-acetate, prednisone, hydrocortisone 17-valerate, hydrocortisone 17-butyrate, betamethasone valerate, triamcinolone acetonide, flucinonide, desonide, flucinolone acetonide, dexamethasone, dexamethasone 21-phosphate, prednisolone, prednisolone 21-phosphate, haloprednone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone, flumethasone pivalate, flunisolide acetate, flucortolone, fluorometholone, fluperolone acetate, fluprednisolone, fluprednisolone valerate, meprednisone, methyl prednisolone, paramethasone acetate, prednisolamate, prednival, triamcinolone, triamcinolone hexacetonide, cortivazol, formocortal, nivazol, and methylprednisone.

Examples of preferred non-steroidal anti-inflammatories include diclodenac, diflunisal, fenoprofen, flurbiprofen, ibuprofen, indomethacin, ketoprofen, melcofenamate, mefenamic acid, naproxen, phenylbutazone, piroxicam, sulindac, tiaprofenic acid, alcolfenac, desoxysulindac, aspirin, salicylamide, salicyclic acid, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole neocinchophen, nimazole, proxazole citrate, tesicam, tesimide, tramadol, triflumidate and tolmetin. Non-limiting examples of particularly preferred non-steroidal anti-inflammatories include ketorolac, ibuprofen, nabumetone, diclofenac, etodolac, and piroxicam.

Preferred pain relief compositions as described above are formulated to facilitate transdermal delivery. For example, the active components of the pain relief composition are preferably in a skin-permeable form. Pain relief compositions may be incorporated into an ointment, cream or paste suitable for topical administration by formulating the components with a suitable carrier as described as U.S. Pat. No. 5,900,249, which is hereby incorporated by reference in its entirety and particularly for the purpose of describing topical formulations. The pain relief compositions described above are preferably formulated with a component that enhances the penetration of the drugs through the skin. It is believed that penetration enhancers facilitate transfer of the drug components through the stratum corneum and into the dermis to provide a local effect. For a discussion of use of penetration enhancers in topical formulations see generally, PERCUTANEOUS PENETRATION ENHANCERS (Eric W. Smith & Howard I. Maibach eds. 1995); Ghosh, T. K. et al. 17 PHARM. TECH. 72 (1993); Ghosh, T. K. et al. 17 PHARM. TECH. 62 (1993); Ghosh, T. K. et al. 17 PHARM. TECH. 68 (1993), all of which are hereby incorporated by reference in their entireties. Preferred penetration enhancers are pharmacologically inert, non-toxic, and non-allergenic, have rapid and reversible onset of action, and are compatible with the pain relief compositions. Non-limiting examples of penetration enhancers include transcutol P, ethyl alcohol, isopropyl alcohol, lauryl alcohol, salicylic acid, octolyphenylpolyethylene glycol, polyethylene glycol 400, propylene glycol, N-decylmethylsulfoxide, DMSO and the azacyclo compounds, as disclosed in U.S. Pat. Nos. 4,755,535; 4,801,586; 4,808,414; and 4,920,101, all of which are hereby incorporated by reference in their entireties and particularly for the purpose of describing penetration enhancers. Preferably, the penetration enhancer is ethyl alcohol. Preferably, the penetration enhancer also functions as a solvent and carrier for the pharmaceutically active ingredients.

More preferably, pain relief compositions are contained in a transdermal delivery patch that is suitable for application to the skin. The transdermal delivery patch preferably comprises at least the pain relief composition and a covering layer that permits the patch to be placed on the area of skin to be treated. Preferably, the transdermal delivery patch maximizes drug delivery through the stratum corneum and into the epidermis or dermis, and minimizes absorption of the pain relief composition into the circulatory system, reduce lag time, promotes uniform absorption, and reduce mechanical rub-off. Preferably, the mechanical patch components conform to the skin during movement to provide comfort and prevent undue shear and delamination.

Preferred transdermal delivery patches comprising the pain relief compositions have advantages over conventional methods of administration. One advantage is that the dose is controlled by the patch's surface area. Other advantages of patches are relatively constant rate of administration, longer duration of action (the ability of to adhere to the skin for 1, 3, 7 days or longer); improved patient compliance, non-invasive dosing, and reversible action (i.e., the patch can simply be removed). Examples of preferred transdermal delivery patches suitable for use with the pain relief compositions include (1) the matrix-type patch; (2) the reservoir-type patch; (3) the multi-laminate drug-in-adhesive type patch; (4) the monolithic drug-in-adhesive type patch; and (5) hydrogel patch; see generally Ghosh, T. K.; Pfister, W. R.; Yum, S. I. Transdermal and Topical Drug Delivery Systems, Interpharm Press, Inc. p. 249-297, hereby expressly incorporated herein by reference). These patches are well known in the art and available commercially.

In a preferred embodiment, a pain relief composition as described above is contained in a reservoir-type transdermal delivery patch. The reservoir-type patch is characterized by a backing film coated with an adhesive and a reservoir compartment comprising a pain relief composition as described above (see, e.g., U.S. Pat. No. 4,615,699, which is hereby incorporated by reference in its entirety and particularly for the purpose of describing transdermal delivery patches that comprise a reservoir). The transdermal delivery patch may contain a single compartment or multiple compartments. The adhesive-coated backing layer preferably forms a margin around the reservoir's boundaries to provide a secure seal with the skin and hold the reservoir adjacent to the skin. Preferably, the adhesive margin has a width in the range of about 0.25 cm to about one cm. The reservoir-type patch preferably comprises a membrane with a degree of porosity of about 3 to about 30 percent. The volume and surface area of the patch may be adjusted depending on the application. The volume is preferably in the range of about 0.2 mL to about 2.0 mL, and the surface area of the membrane is preferably in the range of from about 5 cm² to about 40 cm². Within the pain relief composition in the reservoir, the concentration of each of the active components (opioid agonist, N-methyl-D-aspartate receptor antagonist and anti-inflammatory) is preferably in the range of about 0.25 percent to about 50 percent, by weight based on the total weight of the composition in the reservoir. For any particular reservoir-type patch, the concentration of each of the active components is preferably adjusted to provide the desired dosage to the patient when the patch is applied to the skin, taking into account the porosity of the membrane, the surface area of the patch, the efficiency of the penetration enhancer, and potency of the active component, as determined by routine experimentation. For example, typical amounts of various active components (on a per patch basis) are as follows: the amount of fentanyl is preferably in the range of about 1.5 mg to about 15 mg; the amount of sufentanil is preferably in the range of about 0.15 mg to about 1.5 mg; the amount of ketorolac is preferably in the range of about 10 mg to about 180 mg; the amount of dextromethorphan is preferably in the range of about 12 mg to about 36 mg; the amount of dexamethasone is preferably in the range of about 2 mg to about 32 mg; the amount of amantadine is preferably in the range of about 5 mg to about 200 mg; the amount of amitryptiline is preferably in the range of about 30 mg to about 300 mg; the amount of methadone is preferably in the range of about 45 mg to about 180 mg; and the amount of betamethasone is preferably in the range of about 1 mg to about 16 mg.

To facilitate skin permeation, the composition in the reservoir preferably further comprises a carrier in an amount that is effective to dissolve the opioid agonist, N-methyl-D-aspartate receptor antagonist and anti-inflammatory. In a preferred embodiment, the carrier is a penetration enhancer as described above, more preferably ethanol. The amount of carrier in the reservoir is typically adjusted so that the composition in the reservoir has the desired concentration of active components. Typically, the amount of carrier in the reservoir is in the range of about 50% to about 99.5%, by weight based on the total weight of the composition. The composition in the reservoir may further comprise a pharmaceutically acceptable thickening agent to facilitate handling and reduce leakage. A wide variety of pharmaceutically acceptable thickening agents are known to those skilled in the art. Hydroxyethylcellulose (HEC) is an example of a preferred thickening agent. The amount of thickening agent in the composition is preferably in the range of about 0.2% to about 4%, by weight based on the total weight of the composition in the reservoir.

In another preferred embodiment, a pain relief composition as described above is contained in a drug-in-adhesive or hydrogel patch. The monolithic drug-in-adhesive patch design is characterized by the inclusion of the drug formulation in the skin contacting adhesive layer, a backing film and preferably, a release liner. The adhesive functions both to release the anesthetic and adhere the anesthetic matrix to the skin. The drug-in-adhesive system does not require an adhesive overlay and thus the patch size is minimized. Also, drug-in-adhesive type patches are thin and comfortable (see, e.g., U.S. Pat. No. 4,751,087, which is hereby incorporated by reference in its entirety and particularly for the purpose of describing drug-in-adhesive type transdermal delivery patches).

The transdermal delivery patches described above may be manufactured, packaged, stored and labeled according to standard procedures. For example, see the procedures described in Bova et al., Product Development and Technology Transfer for Transdermal Therapeutic Systems in TRANSDERMAL CONTROLLED SYSTEMIC MEDICATIONS 379-396 (Y. W. Chien ed. 1987); J. W. Dohner, Development of Processes and Equipment for Rate Controlled Transdermal Therapeutic Systems in TRANSDERMAL CONTROLLED SYSTEMIC MEDICATIONS 349-364 (Y. W. Chien ed. 1987); H-M Wolf et al., Development of Processes and Technology for Adhesive-Type Transdermal Therapeutic Systems in TRANSDERMAL CONTROLLED SYSTEMIC MEDICATIONS 365-378 (Y. W. Chien ed. 1987), all of which are hereby incorporated by reference in their entireties.

The pain relief compositions are preferably delivered transdermally to humans by applying the composition to the skin, e.g., by applying the ointment, cream, lotion or transdermal delivery patch described above. When a transdermal delivery patch is used to administer a pain relief composition, the dosage to achieve pain relief is typically determined by the active surface area of the medicated portion of the patch in direct contact with the skin. Several dosage strengths are advantageous depending upon the severity of the pain. In general, a physician can begin dosing with a low or intermediate strength patch and then, depending upon the effectiveness, adjust the dosage up or down by prescribing a patch of higher or lower active concentration or a patch of larger or smaller surface area, or, in some cases, multiple patches. In general, the pain relief composition will comprise from about 0.001 percent to about 20 percent by weight of the patch, preferably from about 5 percent to about 25 percent by weight of the patch. For matrix (drug-in-adhesive) type patches, the pain relief compositions preferably comprise from about 0.5 percent to about 20 percent by weight of the patch. For patches comprising a hydrogel, the pain relief compositions preferably comprise from about 0.5 percent to about 10 percent by weight of the patch. Fresh patches may be administered multiple times per day, but, preferably, a fresh patch is administered about every 18 to about every 48 hours, more preferably daily.

### EXAMPLE 1

An empty reservoir patch is obtained from a commercial source. The empty patch has a backing layer (outer layer exposed to environment), a reservoir layer (with compartment having volume of about 0.2 mL), a membrane layer having a surface area of about 7 cm² (to control the flow of the pain reliever composition from the reservoir to the skin), a silicon adhesive layer (to adhere the membrane layer to the skin) and a protective liner (to be peeled from the adhesive layer prior to placement on the skin). The empty patch is also equipped with an injection port to permit the pain reliever composition to be injected into the reservoir layer.

A pain reliever composition is prepared in a laminar flow glove box using sterile technique as follows: A solution having a total weight of about 10 grams is prepared by stirring together about 0.1 grams hydroxyethylcellulose (thickening agent), about 0.123 grams fentanyl (opioid agonist), about 0.125 grams dextromethorphan (NMDA receptor antagonist), about 0.625 grams ketorolac tromethamine (anti-inflammatory), and about 11.8 mL ethanol (USP). The total volume of the resulting viscous solution is about 12.5 mL. A portion of the solution is drawn into a 5 mL syringe using a 16 gauge needle. The 16 gauge needle on the 5 mL syringe is detached and replaced with a 21 gauge needle. The 21 gauge needle is inserted into the injection port of the empty patch and 0.2 mL of the solution is injected into the patch reservoir. The needle is gently removed from the port, and the patch is gently massage to expel any remaining air from the reservoir. The port is then sealed. The resulting reservoir patch contains about 1.96 mg of fentanyl, about 2.0 mg of dextromethorphan, and about 10 mg of ketorolac tromethamine.

### EXAMPLES 2-11

A series of pain reliever compositions and transdermal delivery patches are prepared in the general manner described in Example 1, except that the sizes of the patches and the amounts and types of opioid agonist, NMDA receptor antagonist, and anti-inflammatory are varied as shown in Table 1.

TABLE 1

| | | | NMDA Receptor | NMDA Receptor |
|---|---|---|---|---|
| No. | Patch Size | Opioid Agonist | Antagonist | Antagonist |
| 2 | 7 cm², 0.2 mL | 1.96 mg Fentanyl | 12 mg | 10 mg Ketorolac |
| | | 0.196 mg | Dextromethorphan | |
| | | Sufentanil | | |
| 3 | 14 cm², 0.6 mL | 5.88 mg Fentanyl | 36 mg | 30 mg Ketorolac |
| | | 0.588 mg | Dextromethorphan | |
| | | Sufentanil | | |
| 4 | 21 cm², 0.9 mL | 8.82 mg Fentanyl | 54 mg | 45 mg Ketorolac |
| | | 0.882 mg | Dextromethorphan | |
| | | Sufentanil | | |
| 5 | 28 cm², 1.2 mL | 11.76 mg Fentanyl | 72 mg | 60 mg Ketorolac |
| | | 1.176 mg | Dextromethorphan | |
| | | Sufentanil | | |
| 6 | 35 cm², 1.5 mL | 14.7 mg Fentanyl | 90 mg | 75 mg Ketorolac |
| | | 1.47 mg Sufentanil | Dextromethorphan | |
| 7 | 7 cm², 0.2 mL | 1.96 mg Fentanyl | 15 mg Amantadine | 10 mg Ketorolac |
| | | 0.196 mg | | |
| | | Sufentanil | | |
| 8 | 7 cm², 0.2 mL | 1.96 mg Fentanyl | 30 mg | 10 mg Ketorolac |
| | | 0.196 mg | Amitriptyline | |
| | | Sufentanil | | |
| 9 | 7 cm², 0.2 mL | 1.96 mg Fentanyl | 45 mg Methadone | 10 mg Ketorolac |
| | | 0.196 mg | | |
| | | Sufentanil | | |
| 10 | 7 cm², 0.2 mL | 1.96 mg Fentanyl | 12 mg | 2 mg |
| | | 0.196 mg | Dextromethorphan | Dexamethasone |
| | | Sufentanil | | |
| 11 | 7 cm², 0.2 mL | 1.96 mg Fentanyl | 12 mg | 1 mg |
| | | 0.196 mg | Dextromethorphan | Betamethasone |
| | | Sufentanil | | |

### EXAMPLES 12-15

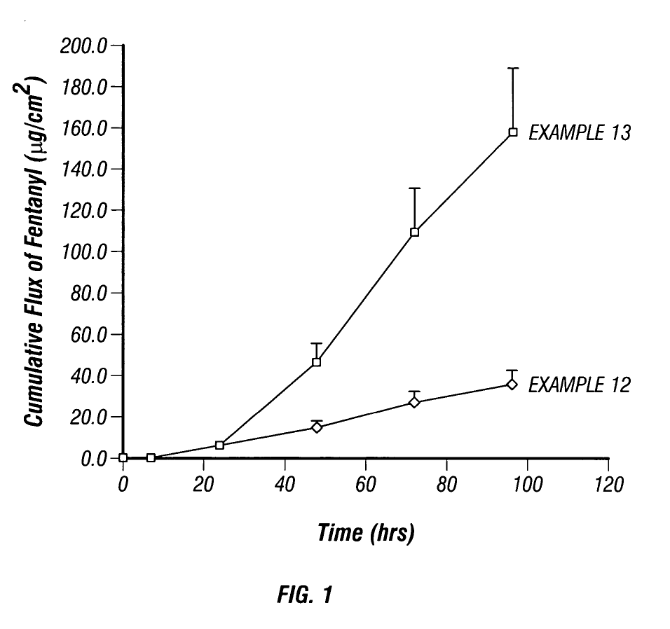

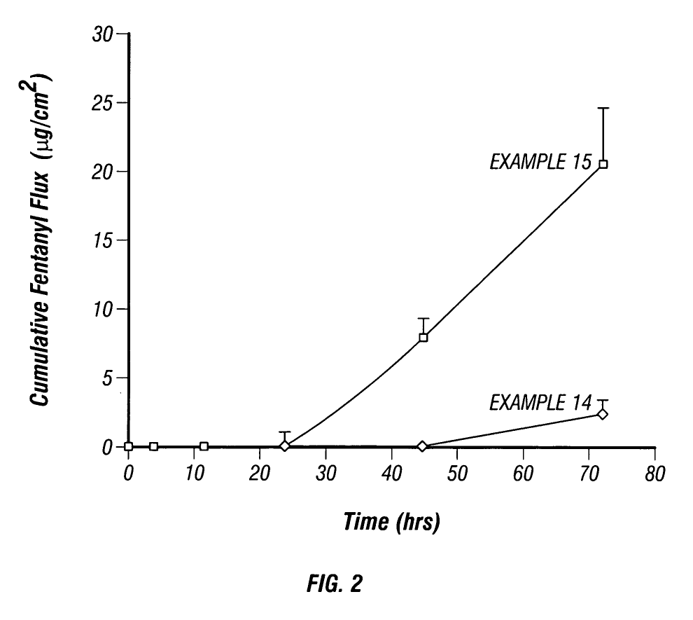

A 1% solution of fentanyl citrate in ethanol was prepared. A separate solution of fentanyl base was prepared by treating a 1% solution of fentanyl citrate with ammonia to raise the pH to 8.5. In vitro flux through human cadaver epidermis was conducted using the Franz cell diffusion method at 32° C. Samples of the receiving buffer solution (PBS pH=7.4) were analyzed by HPLC methods. The donor chambers of the Franz cells contained the samples as shown in Table 2. In vitro flux of the fentanyl citrate and the fentanyl base through the human cadaver epidermis are illustrated by the plots shown in FIGS. 1-2. The in vitro flux data plotted in FIG. 1 shows that the flux of fentanyl base through the cadaver epidermis is higher than the flux of fentanyl citrate. The in vitro flux data plotted in FIG. 2 shows that the flux of fentanyl base is also higher than the flux of fentanyl citrate when measured through both the cadaver epidermis and an ethylene vinyl acetate (EVA) membrane applied to the cadaver epidermis.

TABLE 2

| No. | Sample | FIG. |
|---|---|---|
| 12 | 1% fentanyl citrate/ethanol in direct contact with | 1 |
| | human cadaver epidermis | |
| 13 | 1% fentanyl base/ethanol in direct contact with | 1 |
| | human cadaver epidermis | |
| 14 | 1% fentanyl citrate/ethanol placed on 9% | 2 |
| | EVA membrane attached to human cadaver | |
| | epidermis using silicone adhesive | |
| 15 | 1% fentanyl base/ethanol placed on 9% EVA | 2 |
| | membrane attached to human cadaver epidermis using | |
| | silicone adhesive | |

### EXAMPLE 16

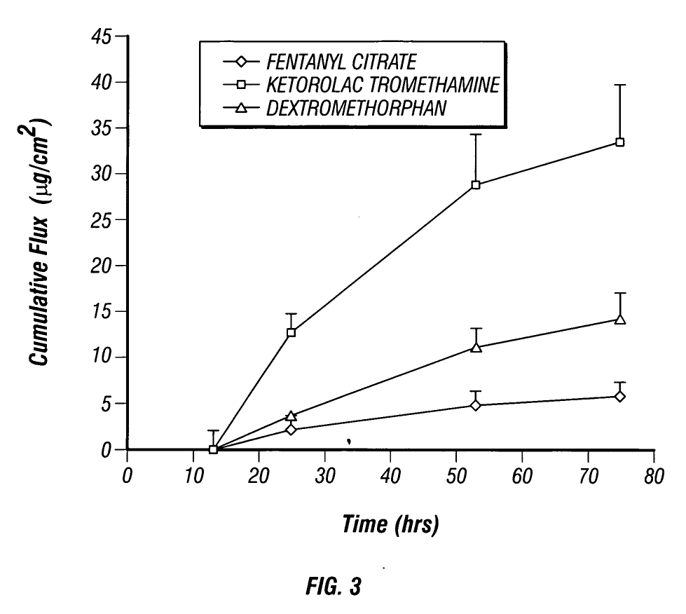

An empty reservoir patch as described in Example 1 was obtained and loaded with an ethanolic solution of 1% fentanyl citrate, 1% ketorolac tromethamine and 1% dextromethorphan. The patch was applied to human cadaver epidermis and in vitro flux was measured as described in Examples 12-15. The results shown in FIG. 3 demonstrate that a reservoir patch may be used to effectively deliver all three components through human skin.

Although the foregoing invention has been described in terms of certain preferred embodiments, other embodiments will become apparent to those of ordinary skill in the art in view of the disclosure herein. Accordingly, the invention is not intended to be limited by the recitation of preferred embodiments, but is intended to be defined solely by reference to the appended claims.

What is claimed is:

1. A composition comprising pharmaceutically effective amounts of
(a) an opioid agonist;
(b) an N-methyl-D-aspartate receptor antagonist different from the opioid agonist; and
(c) an anti-inflammatory, the anti-inflammatory being different from the opioid agonist and different from the N-methyl-D-aspartate receptor antagonist;
wherein the opioid agonist, the N-methyl-D-aspartate receptor antagonist and the anti-inflammatory are each in a skin-permeable form; and
wherein the composition is free of a pharmaceutically effective amount of an α3β4 nicotinic receptor antagonist.

2. The composition of claim 1 in which the opioid agonist is selected from the group consisting of fentanyl, sulfentanil, hydromorphone, oxymorphone, hydrocodone, oxycodone, morphine, methadone, meperidine, ketamine, and propoxyphene.

3. The composition of claim 1 in which the opioid agonist is selected from the group consisting of fentanyl and sulfentanil.

4. The composition of claim 3 in which the opioid agonist is in the form of a pharmaceutically acceptable salt.

5. The composition of claim 1 in which the N-methyl-D-aspartate receptor antagonist is selected from the group consisting of dextromethorphan, amitriptyline, amantadine, ketamine, methadone, and D,L-2-amino-5-phosphono valeric acid.

6. The composition of claim 1 in which the N-methyl-D-aspartate receptor antagonist is in the form of a pharmaceutically acceptable salt.

7. The composition of claim 1 in which the anti-inflammatory is in the form of a pharmaceutically acceptable salt.

8. The composition of claim 1 in which the anti-inflammatory is a nonsteroidal anti-inflammatory.

9. The composition of claim 8 in which the nonsteroidal anti-inflammatory is selected from the group consisting of ketorolac, ibuprofen, nabumetone, diclofenac, etodolac, and piroxicam.

10. The composition of claim 1 in which the opioid agonist is selected from the group consisting of fentanyl and sufentanil, the N-methyl-D-aspartate receptor antagonist is dextromethorphan, and the anti-inflammatory is ketorolac.

11. A transdermal delivery patch comprising the composition of claim 1.

12. The transdermal delivery patch of claim 11 further comprising a skin permeation enhancer.

13. The transdermal delivery patch of claim 11, the composition comprising sufentanil, dextromethorphan, and ketorolac.

14. The transdermal delivery patch of claim 11 that comprises a reservoir.

15. The transdermal delivery patch of claim 14 in which the reservoir comprises a plurality of compartments.

16. The transdermal delivery patch of claim 14 in which the reservoir comprises the composition of claim 1 in which the opioid agonist is selected from the group consisting of fentanyl and sufentanil, the N-methyl-D-aspartate receptor antagonist is dextromethorphan, and the anti-inflammatory is ketorolac.

17. A method for treating pain, comprising identifying a human suffering from pain and applying the transdermal delivery patch of claim 11 to the human.

18. The method of claim 17, in which the pain is chronic pain.

19. A transdermal delivery device comprising the composition of claim 1 and means for delivering the composition transdermally to a human.

20. The transdermal delivery device of claim 19 in which the means for delivering the composition transdermally to the human comprises the transdermal delivery patch of claim 11.
